(19) United States
(12) Patent Application Publication
(10) Pub. No.: US 2016/0166712 A1
(43) Pub. Date: Jun. 16, 2016

(54) MURINE WOUND MODEL FOR TESTING PATHOGEN VIRUENCE AND THERAPEUTIC EFFICACY

(71) Applicants: Rebecca L. Pavlicek, Bremerton, WA (US); Daniel V. Zurawski, New Market, MD (US); Mitchell G. Thompson, Washington, DC (US); Chad C. Black, Knokville, TN (US)

(72) Inventors: Rebecca L. Pavlicek, Bremerton, WA (US); Daniel V. Zurawski, New Market, MD (US); Mitchell G. Thompson, Washington, DC (US); Chad C. Black, Knokville, TN (US)

(21) Appl. No.: 14/570,424

(22) Filed: Dec. 15, 2014

(51) Int. Cl. A61K 49/00; G01N 33/50; C12Q 1/68

(52) U.S. Cl. CPC A61K 49/0008; C12Q 1/689; G01N 33/5091

(57) ABSTRACT

The invention provides a murine model of wound infection and biofilm formation and methods for producing such model. The inventive murine model of wound infection may be used to evaluate pathogen virulence and test efficacy of a pharmaceutical composition and/or a treatment procedures in preventing or treating wound infection, reducing biofilm formation, or reducing symptoms in a subject.

### CROSS-REFERENCE OF RELATED APPLICATIONS

This application claim priority to U.S. Provisional Application No. 61/915,858 filed Dec. 13, 2013.

### FIELD OF THE INVENTION

This invention is related to wound treatment. More particularly, the invention relates to a murine wound model to study wound infection and pathogen virulence. The invention is also related to methods of producing the same and using it as an animal model for testing novel therapeutic treatment strategies and therapeutic agents for treating human wound infection.

### BACKGROUND

Despite significant advancements in wound and burn care, wound infections caused by multidrug-resistance (MDR) microorganism, including Acinetobacter baumannii, still remain a major problem with regards to morbidity and mortality in both civilians and wounded military service members (1, 2, 6, 7). Patients' recovery from traumatic injuries often require weeks to months-long hospitalization, increasing the risk for wound and surgical site infection caused by ESKAPE pathogens, is a fraction of antibiotic-resistant bacteria capable of escaping biocidal action of antibiotics, such as Enterococcus faecium, Staphylococcus aureus, Klebsiella pneumoniae, Acinetobacter baumannii, Pseudomonas aeruginosa and Enterobacter spp. Acinetobacter baumannii, has become a problematic, nosocomial species of bacteria responsible for various types of infection ranging from urinary tract infection to skin and soft tissue infections as well as ventilator-associated pneumonia (VAP), where all of these infections predominantly arise in immunocompromised patients (1, 2). Complications in treatment have grown in recent years primarily due to the bacteria's increased resistance to antibiotic treatment, which are often characterized as multidrug-resistance (MDR), extensively drug resistance (XDR) or PAN-resistant (3). For example, although wound infections caused by A. baumannii is less appreciated in clinical setting, as the mortality rate is not as high as is associated with VAP or cases of septicemia (4, 5). It represents a large fraction of the clinical indications observed (1, 2, 4), and need to be considered when testing novel antimicrobials against bacterial pathogen.

Wound infections occur at higher rates with military service members possibly due to issues associated with the type of traumatic injury, time until treatment, and the fact that they will pass through multiple medical facilities before arriving at a U.S. treatment facility (6). In the civilian sector, A. baumannii has been found to be associated with pressure ulcers and wounds in diabetics (8, 9), and was even implicated in a case of necrotizing fasciitis (10). As new treatments and therapeutics are developed for A. baumannii and other MDR-bacteria, an effective animal model is required to evaluate these treatments and indications in order to generate the required preclinical data before moving onto human trials.

Animal models have been developed to evaluate the virulence of ESKAPE pathogens such as A. baumannii, and efficacy of treatment against them. Both murine and rat pulmonary models of infection have been developed, which relied upon mucin (11), cyclophosphamide (12), or morphine (13) to establish the infection. Others used diabetic mice (14 and WO 2004060476) to study of systemic A. baumannii infection. Russo et al. (15) have reported a rat pulmonary models of infection as well as a soft-tissue infection model using an abscess model of infection (15). Other small rodent model utilized intravenous inoculums to induce sepsis and evaluate treatments against A. baumannii infection (16, 17, 18). However, while there are existing animal models for the study of wound infection, these animal models primarily relied on burn injuries and offers short wound evolution windows (19, 20, 21), typically between 24-72 hours. This window is not long enough to allow adequate evaluations of potential therapeutics or treatment strategies, and cause the results to be less predictive of the real efficacy. Currently, there is no murine cutaneous wound model, which offers long duration of wound infection, so antimicrobials can be evaluated from multiple quantitative/qualitative and microbiological/wound healing endpoints.

### DETAILED DESCRIPTION OF FIGURES

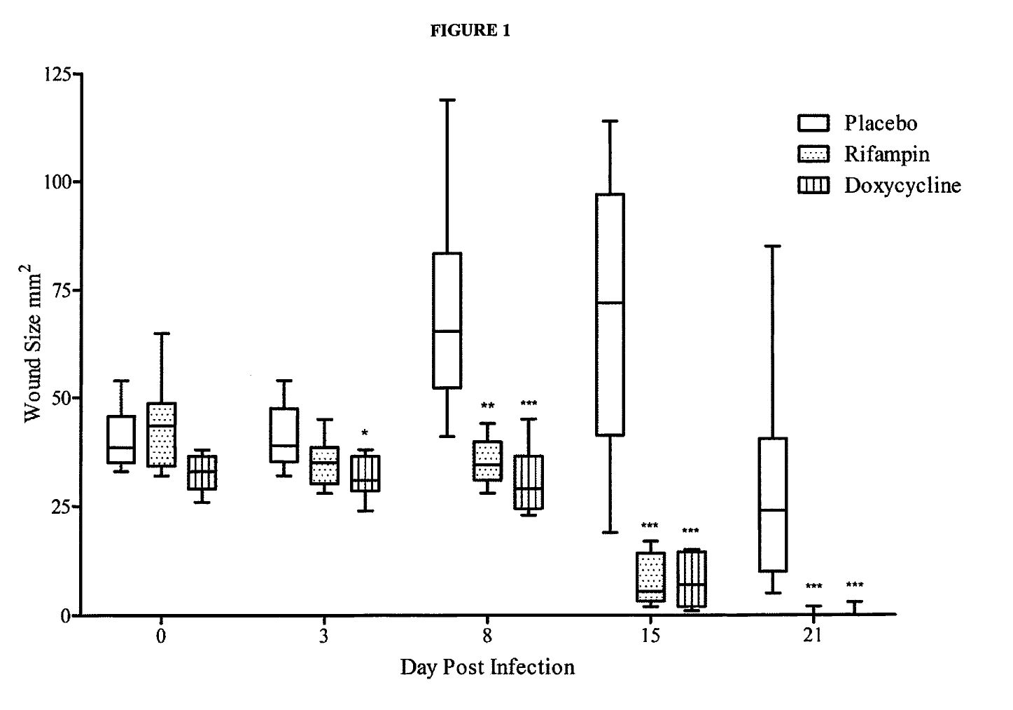

FIG. 1: Box and Whisker plots of wound sizes on days 0, 3, 8, 15 and 21 post-infection. Boxes show median and interquartile ranges, while whiskers represent 95% CI. Groups were compared each day via Two-way Repeated Measures ANOVA followed by a Bonferroni post-test; *, **, and *** represent p<values of 0.05, 0.01, and 0.001 respectively.

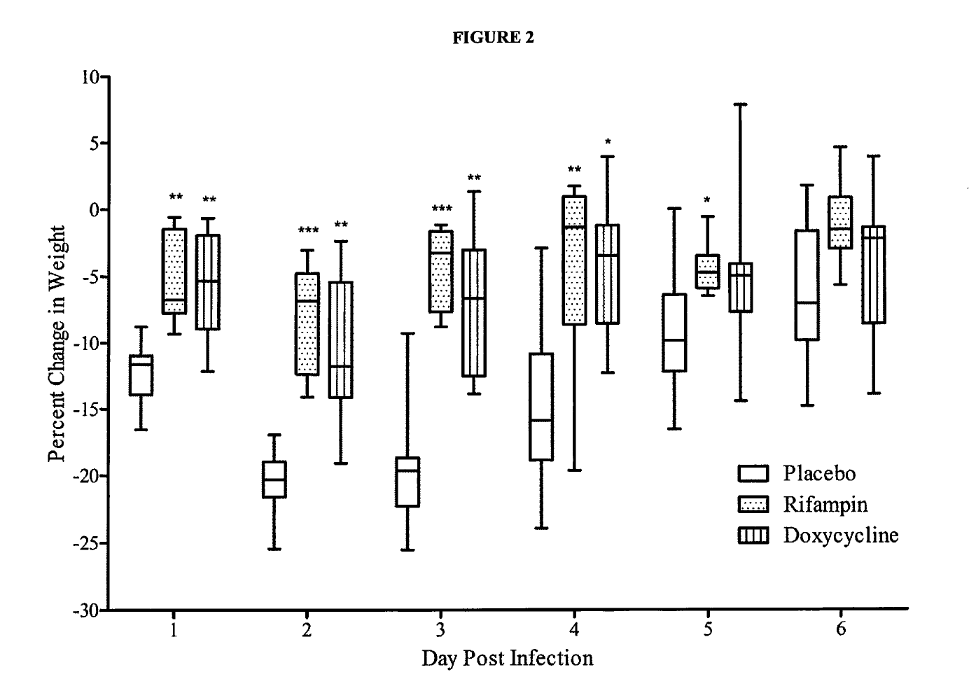

FIG. 2: Box and Whisker plots of percent weight change on days 1 through 6 post-infection. Boxes show median and interquartile ranges, while whiskers represent 95% CI. Groups were compared each day via Two-way Repeated Measures ANOVA followed by a Bonferroni post-test; *, **, and *** represent p<values of 0.05, 0.01, and 0.001 respectively.

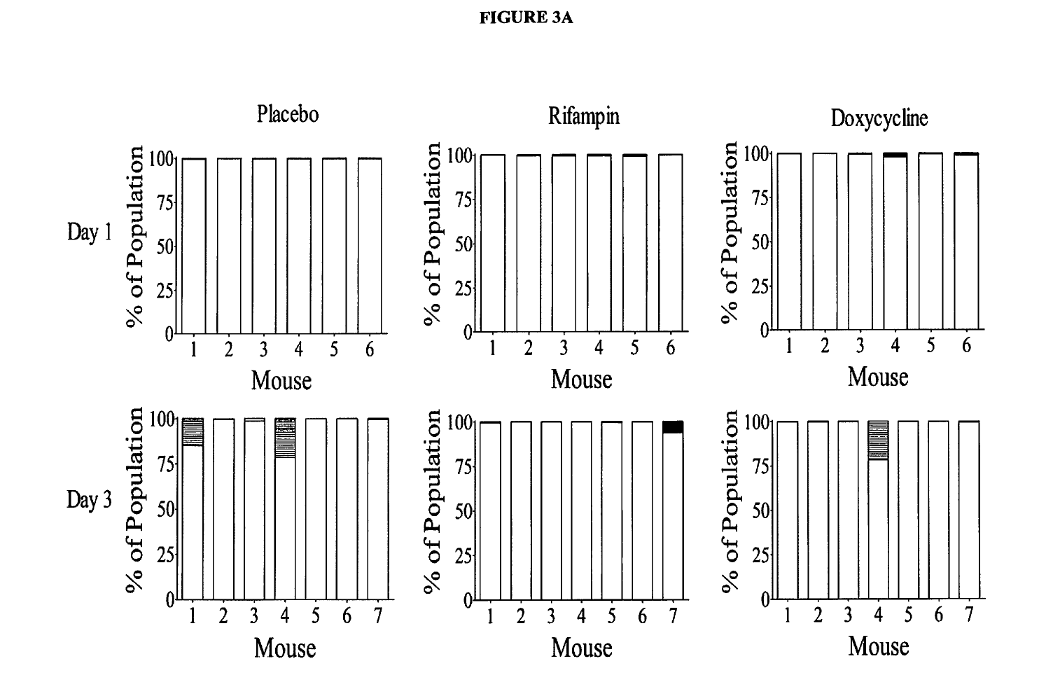

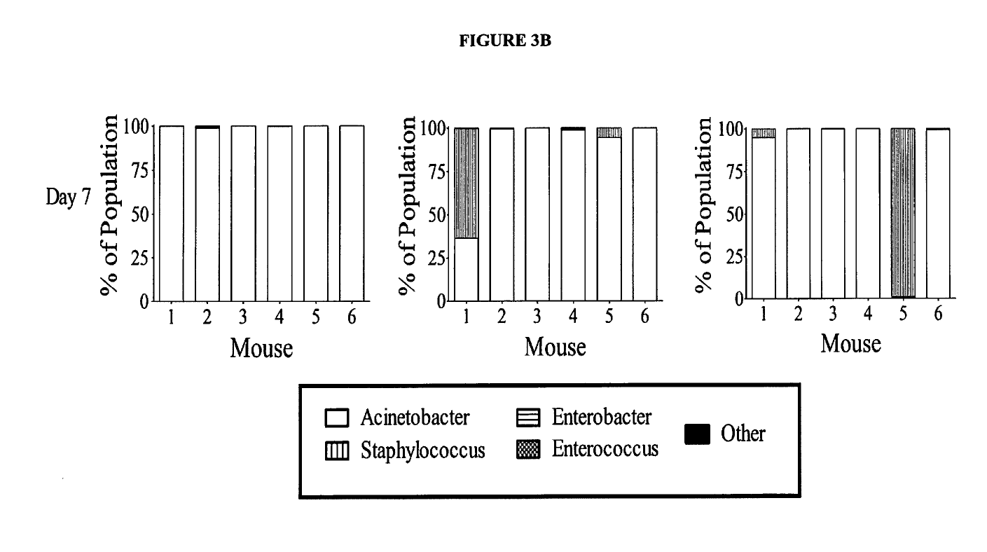

FIG. 3: Bar graph represent bacterial constituents by percentage within the wound of a single mouse in treatment groups. Rows of graphs represent Days 1, 3, 7 post-infection respectively, while columns represent treatment with placebo, rifampin 25 mg/kg/day, or doxycycline 50 mg/kg/day.

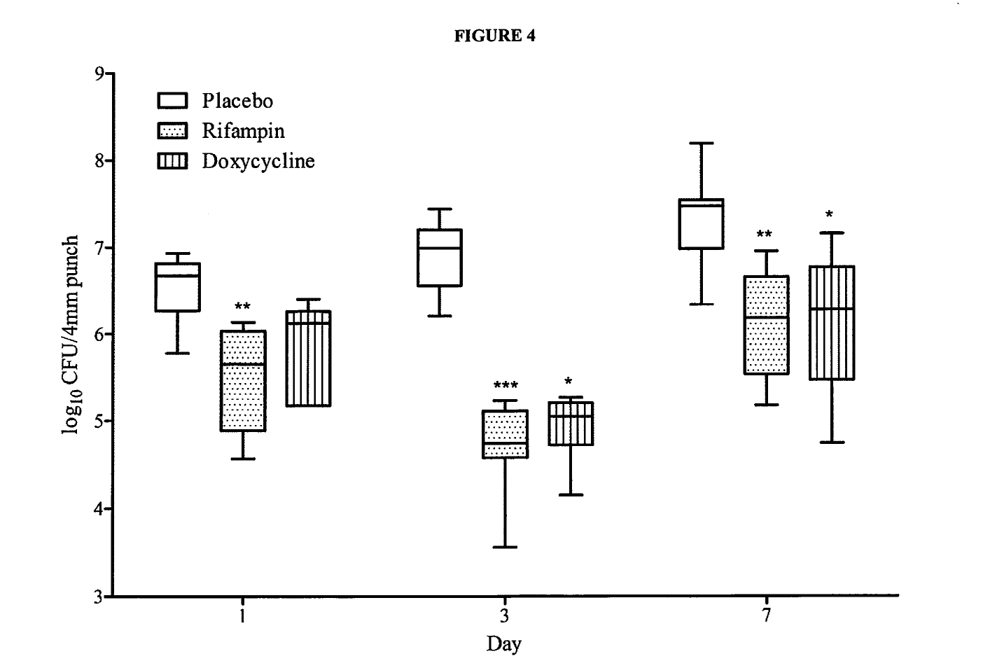

FIG. 4: Box and Whisker plots of CFUs recovered from 4 mm biopsy punches from the wound bed on Days 1, 3, 7. Boxes show median and interquartile ranges, while whiskers represent 95% CI. Treatments were compared via Kruskal-Wallis test followed by Dunn's multiple comparisons post-test; *, ** and *** represent P<values of 0.05, 0.01 and 0.001 respectively.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates. Unless specifically defined, all technical and scientific terms used herein have the same meaning as commonly understood by a skilled artisan in biochemistry, cellular biology, molecular biology, and the medical sciences (e.g., dermatology, etc.). All methods and materials similar or equivalent to those described herein can be used in the practice or testing of the present disclosure, with suitable methods and materials being described herein.

### DEFINITIONS

Articles “a” and “an” are used herein to refer to one or to more than one (i.e. at least one) of the grammatical object of the article. Articles “a” and “an” are used herein to refer to one or to more than one (i.e. at least one) of the grammatical object of the article.

“Immunocompromised”, describes an individual who is undergoing immunosuppression, or whose immune system is weakened for other reasons (for example, chemotherapy, HIV, and Lupus).

“Immunosuppressive drugs” are any molecules that interfere with the immune system and blunt its response to foreign or self antigens. This term used herein is intended to encompass any drug or molecule that can induce neutropenia in a subject, which include but not limited to cyclophosphamide (CYC), azathioprine (AZA), cyclosporine A (CSA), and mycophenolate mofetil (MMF), prednisone, methyl prednisolone, monoclonal antibodies against T cells, antilymphocyte globulin and antithymocyte globulin.

“ESKAPE pathogens” is a faction of antibiotic-resistant bacteria capable of “escaping” biocidal action of antibiotics and mutually respecting new paradigms in pathogenesis, transmission and resistance, which include but not limited to Enterococcus faecium, Staphylococcus aureus, Klebsiella pneumoniae, Acinetobacter baumannii, Pseudomonas aeruginosa and Enterobacter spp. (47).

“Neutropenia” is a condition characterized by an abnormally low number of neutrophils, which serve as the primary defense against infections by destroying bacteria in the blood.

“Therapeutic” is a treatment, a therapy, a drug, an antimicrobial agent, or a vaccine.

As used herein, the term “Murine” is defined of or relating to rodents which includes but not limited to, rats, mice, hamsters, gerbils and the like.

As used herein, the term “mouse” includes but not limited to rats, mice, hamsters, gerbils and the like. Other suitable animals for the present invention may also include rabbits, cats, and dogs.

As used herein, the term “administer” or “administering” refers to providing, contacting, and/or delivery of a therapeutic (e.g., a therapeutic being tested for wound healing or antimicrobial properties) by any appropriate route to achieve the desired effect. These therapeutics may be administered to a subject in numerous ways including, but not limited to, orally, ocularly, nasally, intravenously, topically, as aerosols, suppository, etc. and may be used in combination.

As used herein, the term “therapeutically effective” refers to a dosage of a therapeutic (e.g., a therapeutic being tested for wound healing or antimicrobial properties) that is effective for eliciting a desired effect. This term as used herein may also refer to an amount effective at bringing about a desired in vivo effect in an animal, mammal, or human. A therapeutically effective amount may be administered in one or more applications or dosages and is not intended to be limited to a particular formulation, combination or administration route. It is within the scope of the present disclosure that the therapeutic may be administered at various times during the course of wound healing. The times of administration and dosages used will depend on several factors, such as the goal of treatment (e.g., treating v. preventing), condition of the subject, etc. and can be readily determined by one skilled in the art. In certain embodiments, the agent may be in the form of a pharmaceutical composition.

As used herein, the term “pharmaceutical composition” refers to the combination of a therapeutic (i.e., for example, a therapeutic being tested for wound healing or antimicrobial properties) with a carrier, inert or active, making the composition especially suitable for diagnostic or therapeutic use in vivo or ex vivo. A “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (48).

The present disclosure addresses the shortcomings of current small animal models by providing a murine model to study wound infection. Methods of using and producing this model are also disclosed. Such uses may include, but are not limited to, 1) studying pathogen virulence associated with wound infection; 2) testing mechanisms or actions or developing new wound healing therapeutics, such as an antimicrobial drug, a vaccine or a treatment strategy.

An aspect of the presentation invention is a method for producing a murine wound model, which offers longer duration of infection while using smaller inoculum of pathogen. The longer infection widow allows antimicrobials or other therapeutics to be evaluated from multiple quantitative and qualitative microbiological and/or wound healing endpoints. The use of diminutive inoculum reduces the loss of experimental animals due to aggressive infection. The smaller inoculum dose also closely resembles the physiological conditions of human infection process. Individuals who develop wound infections normally do not get inoculated with a high dose of a pathogen but was rather introduced to a small inoculum, which then multiplies under suitable condition.

According to this invention, a method of producing a murine wound model, comprises a) administering to a mouse an therapeutically effective amount of an immunosuppressive drug 1-5 days prior to the inoculation of a pathogen, which is associated with wound infection; b) creating one or more cutaneous wound on the mouse; c) inoculating the wound site with an virulent strain of said pathogen; and d) allowing the pathogen to multiply.

The immunosuppressive drug used in this invention is designed to induce a temporary neutropenia in the mouse, which depletes circulating neutrophils before inoculation to establish an infection. Prior study has shown that the mouse's full immune system can recover 3 to 4 days after the last administration of the immunosuppressive drug allowing a evaluation of efficacy of potential antimicrobials. The immunosuppressive drug may be used in this invention include but not limited to cyclophosphamide (CYC), azathioprine (AZA), cyclosporine A (CSA), and mycophenolate mofetil (MMF), prednisone, methyl prednisolone, monoclonal antibodies against T cells, antilymphocyte globulin and antithymocyte globulin (49,50). Suitable routes of administration may include, for example, oral, rectal, transmucosal, transnasal, intestinal or parenteral delivery, intramuscular, subcutaneous and intramedullary injections as well as intrathecal, direct intraventricular, intracardiac, into the common coronary artery, intravenous, inrtaperitoneal, intranasal, or intraocular injections. The administration may be carried out 1-5 days prior to pathogen inoculation.

Following immunosuppressant administration, one or more cutaneous wound is created for pathogen inoculation. In one embodiment, cutaneous wound were created via full dermal puncture. The cutaneous wound sites have advantages over the burn model because it permits full pathogen exposure to the blood stream, allows assessment of both systemic and topical treatments. It is designed to closely mimic the wounds observed in trauma settings. After inoculation, the wound may be covered by a dressing to promote scab formation while keeping the wound site moist, which also improves biofilm formation.

The pathogens inoculants contemplated in this invention are of diminutive volume and may comprises bacteria or fungal agents known to be associated with wound infection, which includes but not limited to bacteria such as the ESKAPE pathogens, Escherichia coli, Stenotrophomonas maltophilia and selective Streptococcus and Corynebacterium species, and fungal agents such as Candida albicans and certain Aspergillus and Mucorales species. Routine inoculation routes of inoculation may include oral, rectal, transmucosal, especially transnasal, intestinal or parenteral delivery, including intramuscular, subcutaneous and intramedullary injections as well as intrathecal, direct intraventricular, intracardiac, into the common coronary artery, intravenous, inrtaperitoneal, intranasal, or intraocular injections. In an embodiment of this invention, the pathogen is inoculated topically prior to occlusive dressing, or through the occlusive dressing. Pathogen inoculation may be performed in one or more doses.

In a preferred embodiment of this invention, a method of producing a murine wound infection model, comprises 1) administering Cyclophosphamide treatment on Day 4 (150 mg/kg) and Day −1 (100 mg/kg) prior to inoculation of a wound infection pathogen via intraperitoneal (IP) injection; 2) creating a cutaneous wound on the mouse; 3) inoculating the wound site with A. baumannii AB5075; 4) covering the wound with dressing for 7 days.

The inventive animal model may be used to determine the virulence of a wound infection pathogen, including discriminating between the inherent differences in virulence of various clinical isolates. A method to assess the virulence of a wound infection pathogen, comprising 1) producing a murine wound model for a selected pathogen is as previously described; 2) determining the virulence of the pathogen. Tests may be used to determine the virulence of the pathogen includes but not limited to routine qualitative and quantitative measures such as length of the infection (i.e. time for wound closure), and/or bacteria load.

The inventive animal model may be also used to evaluate the efficacy of a potential wound healing therapeutics, such as an antimicrobial, a drug, a vaccine or a wound treatment. The method for evaluating the efficacy of a wound healing therapeutics comprising 1) producing a murine wound model for wound infection as previously described; 2) administering an pharmaceutically effective amount of a wound healing therapeutic to the animal; 3) evaluating efficacy of said wound healing therapeutic against said wound infection pathogen. The efficacy a wound therapeutic may be evaluated using one or more routine qualitative and quantitative assessments, such as 16S analysis, PNA-fish analysis, biofilm study by SEM analysis, colony formation measurement (colony forming units or CFU), or wound size assessment over time.

### Example 1

### Producing a Murine Wound Model

Bacterial Strain and Inocula Preparation

A. baumannii clinical isolate AB5075 was used in all experiments. In prior studies, several known effective antimicrobials were evaluated against a previously characterized clinical isolate of A. baumannii, AB5075. It is demonstrated that this strain causes severe clinical disease in a murine pulmonary model, and has a high level of resistance to most clinically used antibiotics (22). With the introduction of AB5075 in a neutropenic murine excision model, a traumatic wound infection can be successfully simulated, and novel antimicrobial compounds were evaluated as a part of future clinical treatment. Therefore, it is selected for this embodiment.

AB5075 was isolated from a patient from Walter Reed Army Medical Center between 2008 and 2009 (23). A. baumannii strain was chosen that was virulent enough to cause an infection with a small inoculating dose. For this purpose, AB5075 was chosen for this model as previous study has shown this strain is more virulent than the other A. baumannii isolates that were tested (22). AB5075 is shown to be extensively drug resistant (22), but susceptible to both rifampin (MIC=4.0 μg/mL) and doxycycline (MIC=0.125 μg/mL). Because the antibiotic susceptibilities of AB5075 is known (22), the wound model may be used for validation of treatment using IP injected doxycycline and rifampin. AB5075 was propagated on Lennox Luria-Bertani (LB) media (Becton, Dickinson and Co., Sparks, Md.) for all experiments. To prepare inocula for animal infection, 100 μl of AB5075 overnight culture was subcultured into 10 mL of LB, and then grown at 37° C. and shaking at 250 rpm in a 250 ml Erlenmeyer flask. Cells in mid-log growth phase were harvested when the culture grew to an OD₆₀₀ of 0.7. Cells were washed twice with sterile phosphate buffer saline (PBS), and then resuspended in PBS so that 25 μL of the suspension contained 5.0×10⁴ cells. The cell concentration of the suspension was verified via a Petroff-Hauser counting chamber prior to inoculation of mice, and confirmed by serial dilution and plating on LB agar using a spiral plating system (AUTOPLATE®, Advanced Instruments, Inc., Norwood, Mass.).

### Preparation of Immunosuppressive Drug

Neutropenic agent cyclophosphamide was purchased in powder form from Baxter (Deerfield, Ill.), and dissolved in sterile 0.9% sodium chloride injection solution (Hospira Inc., Lake Forest, Ill.) to a final concentration of 10 mg/mL. Antibiotics rifampin and doxycycline were purchased in powdered form from Sigma-Aldrich (St. Louis, Mo.), and dissolved in 0.9% sterile sodium chloride injection solution to final concentrations of 20 mg/mL. All treatments were kept on ice or refrigerated until use.

### Mice and Husbandry

Female BALB/c mice were purchased from the National Cancer Institute, Animal Production Program (Frederick, Md.). Female BALB/c mice were chosen because BALB/c mice are skewed to a Th2 immune response (34, 35), and this immune response favors the establishment of infection by the gram negative ESKAPE (36) pathogens (Mark Shirtliff, personal communication). The mice used in these experiments were six to ten weeks of age and weighed 14 to 20 g. All mice received sterile food and water ad libitum and were housed in groups of three, in sanitized cages, and on sterile paper bedding. Dry rodent chow was supplemented with DIETGEL® Recovery (CLEARH₂O®, Portland, Me.) during the 48 hours following wounding.

### Creating Murine Dorsal Wound Model

All procedures were performed in accordance with protocol IB02-10 that was approved by the Walter Reed Army Institute of Research (WRAIR)/Navy Medical Research Center (NMRC) Institutional Animal Care and Use Committee (Silver Spring, Md.). Mice received 150 mg/kg and 100 mg/kg cyclophosphamide intraperitoneal (IP) injections, before wounding and infection, on days −4 and −1, respectively (22, 24). On Day 0, the day of wounding and inoculum, mice were anesthetized with ketamine 130 mg/kg (KETASET®, Fort Dodge Animal Health, Fort Dodge, Iowa), xylazine 10 mg/kg (ANASED®, Lloyd Inc. Shenandoah, Iowa), and buprenorphine 0.05 mg/kg (Hospira Inc., Lake Forest, Ill.) injections. Hair was clipped from the cervical to mid-lumbar dorsum, and the skin scrubbed with iodine solution followed by an ethanol rinse. A 6.0 mm disposable skin biopsy punch (VisiPunch, Huot Instruments, LLC, Menomonee Falls, Wis.) was used to create a full-thickness skin defect overlying thoracic spinal column and the adjacent musculature. Twenty-five μL containing 5.0×10⁴ AB5075 cells in a PBS suspension were pipetted into the wound, and allowed to absorb for three minutes. A circular cutout (30 mm diameter) of transparent dressing (TEGADERM™Roll, 3M Health Care, St. Paul, Minn.) was placed over the wound and secured with tissue adhesive (VETBOND™, 3M Animal Care, St. Paul, Minn.). A cutaneous wound model was selected based on a previous study, where an open wound did not adversely affect animal health in a >15 day protocol (37). However, the size of the wound was increased to 6 mm in order to facilitate subsequent punch biopsies to measure bacterial burden in the wound tissue.

Beginning four hours post-inoculation, mice were treated with either rifampin at 25 mg/kg IP injection once daily, doxycycline 25 mg/kg IP injection twice daily, or an equivalent vehicle control over the course of a six day treatment period (25, 26). On Day 7 the transparent dressing was removed, treatment discontinued, and the wound monitored for closure through Day 25.

### Example 2

### Assessment of Wound Infection/Efficacy of Antibiotics

Quantification of Bacteria within the Wound Bed

To measure viable bacterial cells within the wound, mice were euthanized via ketamine (250 mg/kg) and xylazine (25 mg/kg) overdose according to protocol, and a 4 mm disposable skin biopsy punch (ACUDERM® Inc., Fort Lauderdale, Fla.) was used to remove a disc of wound bed material. The disc was placed in 1.0 mL of sterile PBS in a stomacher bag and manually disrupted. Serial ten-fold dilutions of homogenate were plated via spiral plater (AUTOPLATE®, Advanced Instruments, Inc., Norwood, Mass.) onto eosin methylene blue (EMB) agar (Becton, Dickinson and Co., Sparks, Md.). Plates were incubated overnight at 37° C., and then colony forming units (CPU) were enumerated.

Characterization of Bacterial Community within the Wound Bed

Three mice from each treatment group (placebo, rifampin, and doxycycline) and from representative time points (four hours post-wounding and on Days 1, 3, and 7) were sacrificed for wound bed bacterial community profiling. Wound bed tissue was excised, placed in 250) μL PBS, and frozen at −80° C. Samples were evaluated for bacterial species diversity using pyrosequencing-based analysis of 16S rRNA genes (Research and Testing Laboratories, LLC, Lubbock, Tex.).

The 16S rRNA sequences in FASTA format were analyzed using mothur software (27). For quality filtering sequences that had quality score less than 20 (Q<20), lacked an accurate primer sequence, sequences containing ambiguous characters, and sequences with more than 8 homopolymers were removed. Sequences were then aligned against SILVA (28) alignment template. Sequences that did not map to overlapping regions in the alignment were also removed. Potential chimeric sequences were detected by using Uchime (29) embedded in the mothur software (chimera.uchime) and removed. The high quality sequences were then pre-clustered (30) to further reduce potential influence of sequencing errors. Opertional taxanomic units (OTUs) were determined using average neighbor clustering of sequences with 97% sequence identity. To assess the taxonomic distributions across each sample a weighted representative sequence from each OTU was selected and subsequently was classified using locally running RDP classifier program (50% bootstrap threshold; (31) and the mothur formatted version of the greengenes reference taxonomy (32).

### Quantitative and Qualitative Wound Closure Assessments/Efficacy of Antibacterial Treatment

Wound area measurements were taken on the day of wounding and at subsequent time points using a SILHOUETTE™ wound measurement device (Aranz Medical Limited, Christchurch, New Zealand). Time course wound photographs assessing gross pathology were taken using a five megapixel ISIGHT™ camera (Apple Inc., Cupertino, Calif.).

### Scanning Electron Microscope Evaluation of Wound Bed and Dressing Biofilm

Dressings and wound bed tissue were evaluated by scanning electron microscopy (SEM). A representative mouse from the placebo, rifampin, and doxycycline treatment groups was sacrificed at four hours post-wounding and on days 1, 3, and 7. The transparent dressings and a 4 mm tissue disc for each animal were fixed in 4% formaldehyde, 1% glutaraldehyde, 0.1M PBS. The samples were washed three times using 0.1M PBS, and then post fixed in 1% osmium tetroxide in 0.1M PBS for one hour. The samples were dehydrated in a graded series of ethanol solutions, and then dried (Critical point dryer, Model 28000, Ladd Research Industries, Burlington, Vt.). The samples were mounted by double-sided carbon tape to specimen stubs, and ion coated with gold: palladium (30:70) (Hummer X sputter coater, Anatech Ltd., Alexandria, Va.). The samples were visualized using an Amray 3600 FE scanning electron microscope (Bedford, Mass.) operated at a voltage of 3 KV. Samples were analyzed by scanning ten or more 1000× magnified fields within the wounded tissue and on the portion of the dressing overlying the wounded area. Photomicrographs representative of the observed biofilm density were taken at 2500× magnification.

### Histological and PNA-FISH Examinations of the Wound Bed

Three mice from each treatment group (placebo, rifampin, and doxycycline) and from representative time points (Days 1, 3, 7, 15 and 23) were sacrificed in order to characterize wound model histopathology. The dorsal wounds were removed en bloc by severing the cervical and lumbar spinal column and trimming the tissue >2 cm beyond the wound edge. The tissue was immediately fixed in phosphate-buffered formalin (10%) for >72 h. The bone was then demineralized for 24 hours using Decal Stat™ (Decal Chemical Corp, Tallman, N.Y.), then rinsed with water for 3-5 minutes, and trimmed in a dorsal-ventral plane bisecting the spinal column and placed back into 10% phosphate buffered formalin. The wound tissue specimens were embedded in paraffin, cut in a dorsal-ventral plane bisecting the spinal column, mounted on positively charged glass slides (Colormark Plus, Thermo Scientific, Portsmouth, N.H.), and stained with hematoxylin (Astral Diagnostics, Inc., West Deptford, N.J.) and eosin (Astral Diagnostics, Inc., West Deptford, N.J.) for light microscopic examination.

Wounds were histologically assessed for presence and of dissemination of bacteria, host immune response, indications of wound healing, i.e. the extent of epithelial migration, coverage, maturation, and amount of granulation tissue present within the wound and if wound and associated inflammation extended into underlying vertebrae, spinal cannal, and/or spinal cord.

One mouse from each treatment group (placebo, rifampin, and doxycycline) and from representative time points (Days 1, 3, and 7) were sacrificed in order to characterize the dissemination of AB5075 within wound tissue via peptide nucleic acid fluorescence in situ hybridization (PNA-FISH) (33). Tissue was trimmed at 3 microns. One drop of PNA probe, AdvanDx Acinetobacter PNA CP0050 (AdvanDx, Inc., Woburn, Mass.), was added to each slide and cover slips were applied. Slides were put on a heating block for 90 min at 55° C. in the dark. Slides were then immersed in preheated, 55° C., deionized water for less than 1 min while (AdvanDx, Inc., Woburn, Mass.) the coverslips were removed. Slides were then immersed in preheated, 55° C., AdvanDx 60× Wash Solution (AdvanDx, Inc., Woburn, Mass.) for 30 min. Slides were then overlaid with coverslips using AdvanDx Mounting Medium (AdvanDx, Inc., Woburn, Mass.). Separately, cultures of A. baumannii and Escherichia coli were kept as positive and negative controls, respectively. One drop of AdvanDx GN Fixation (AdvanDx, Inc., Woburn, Mass.) solution was added to a slide. One small drop of culture was added to the fixation solution. The slides were then placed on a heating block for 20 min at 55° C. to fix the smear. One drop of AdvanDx Acinetobacter PNA CP0050 (AdvanDx, Inc., Woburn, Mass.) was added to each slide and cover slips overlaid. Control slides were further prepared as described above.

### Statistical Analyses

All statistical analyses were carried out using Graphpad Prism software. Wound sizes and weight change were analyzed via Two-way Repeated Measures ANOVA followed by Bonferroni post-test. CFU burdens were compared via the Kruskal-Wallis test followed by Dunn's multiple comparison tests. All results were considered significant if p<0.05.

### Results

Wound Infection with A. baumannii in BALB/c Mice

In a pilot study, using cyclophosphamide-treated mice, wound area of wound inoculated with AB5075 or un-inoculated wound were measured over time. In the A. baumannii-infected mice, the wound became infected, and the time to wound closure was delayed at least 5 days when compared to mice that received no A. baumannii inoculum at Day 0.

### Time Course Gross Pathology and Wound Size of Treated and Untreated Wound Infections

The first assessment of the model included wounds that were infected with A. baumannii and had treatment versus no treatment groups, which were compared with respect to gross pathology. Photographs of the dorsal, full-thickness wounds were taken on Days 3, 8, 15, and 21. On Day. Open wound beds without contraction or re-epithelization were observed, and devitalized epidermal tissue peripheral to wound edges was lightly-colored and correlates with post-TEGADERM® removal necrosis and wound expansion. On Day 15, a variation in the wound and the serocellular crust covering the wound was dependent upon treatment, where the treated wounds appeared smaller and less inflamed. On Day 21, the untreated control wound maintains serocellular crust with minimal contractile healing, whereas, in contrast, the treated wounds displayed a contraction of the wound, and the wounds were either fully closed or had less than 5 mm² overlying serocellular crust when measured.

Wounds were measured via Aranz on Days 0, 3, 8, 15, and 21 post-infection. Wound sizes between groups were not statistically different Day 0 when compared via Kruskal-Wallis test. When wounds were measured on Day 3 post-infection the placebo group (n=12) had a median wound size of 39.0 mm², while rifampin-treated (n=8), and doxycycline-treated (n=9) had median wound sizes of 35.0 mm², and 31.0 mm² (p<0.05). By Day 8, one day after the occlusive dressing was removed, the median wound size for placebo-treated mice had increased to 65.5 mm², while the rifampin-treated wound stabilized at 34.5 mm² (p<0.01), and doxycycline-treated mice had the median decrease to 29.0 mm² (p<0.001). On Days 15 and 21 the placebo group had a median wound sizes of 97.0 mm² and 22.0 mm², while the rifampin-treated mice had medians of 5.5 mm² (p<0.001) and 0.0 mm² (p<0.001). Similarly, the doxycycline-treated mice had medians of 2.0 mm² (p<0.001) and 0.0 mm² (p<0.001), which was indicative of the significant differences seen between antibiotic-treated versus the untreated wounds.

### Weight Loss

Aside from gross pathology observations and wound measurements done with the mice, we were also cognizant of measuring clinical signs of infection. One of the signs that showed a significant difference was weight measurement over time. Mice treated with placebo (n=12) lost a median of 11.6% of their infection-day body weight one day post-infection and lost maximally a median of 20.3% by 2 days post-infection (FIG. 2). Rifampin (n=8) and doxycycline (n=9) treated mice only lost 6.7% (p<0.01) and 5.3% (p<0.01), respectively, on Day 1 post-infection, and maximally lost 6.8% (p<0.001) and 11.8% (p<0.01) (FIG. 2). When compared via Kruskal-Wallis test followed by a Dunn's post-test, rifampin-treated mice maintained less weight loss for 5 days post-infection while doxycycline-treated mice remained significantly different for 4 days.

### Time Course Histopathology of Wounds

After seeing significant differences at the gross observation level that suggested an A. baumannii infection had been established and that this infection can be treated. To examine the wound on a cellular level, both standard histopathology and some non-standard methods were used to examine the localization of bacteria and the impact of antibiotic treatment on the bacteria and host healing. Photomicrographs of hematoxylin and eosin (H&E)-stained tissue, dorsal wound longitudinal sections were prepared and evaluated. The Day 7 slides at a 12.5× low-magnification view demonstrated a varying defect in the widths of the wound bed under the TEGADERM®, and all the wounds were devoid of epidermis with significant inflammatory cell infiltrate at the wound edge. At 40× magnification, the Day 15, untreated, control wound is shown lacked re-epithelization and were covered by serocellular crust. In contrast, on Day 15, when comparing rifampin and doxycycline treatments respectively, a re-epithelization over the wound was observed with granulation tissue surrounding the necrotic cellular debris. On Day 23, the untreated control wound is fully re-epithelized, but lacked evidence of contractile healing and maintained evidence of an expansive over-lying serocellular crust. In contrast, the Day 23, rifampin and doxycycline treatments showed signs of contractile healing and re-epithelized wound beds with less over-lying crust and granulation tissue surrounding necrotic cellular debris.

### PNA-FISH Examination of AB5075 Localization in Wound Tissue

In order to understand where the bacteria were localized in animal tissue sections, the wound and dressing using PNA-FISH were evaluated. The untreated control was evaluated on Day 1 at 12.5× magnification H&E and at 40× using Acinetobacter-specific PNA probe-stained photomicrographs. The A-E designations in the larger view of the H&E-stained photomicrographs correlate to individual PNA probe pictures. The green fluorescent probes demonstrate A. baumannii cells at all levels of the wound bed from superficial serocellular crust at the surface to the deep para-spinal musculature. This was in striking contrast to the rifampin treatment at Day 1. The absence of green fluorescence demonstrates a clearance of A. baumannii cells below the level of detection at all levels of the wound bed from superficial serocellular crust to the deep para-spinal musculature.

The doxycycline treatment at Day 1 displayed an intermediate phenotype. As with before, the A-F designations in H&E photomicrograph correlate to the increased magnification of individual PNA probe pictures. Green fluorescent probes demonstrate the presence of A. baumannii cells on the surface of the wound and deep to the sub-dermal fat; however, fluorescent cells are absent in the deep para-spinal musculature, suggesting systemically-delivered (IP injections) doxycycline has less wound bed penetration relative to the rifampin-injected animals.

### Community Profiling of Wounds by 16S Pyrosequencing

The bacterial community composition was determined by 16S rRNA pyrosequencing (Research and Testing Laboratories, LLC, Lubbock, Tex.). Bacterial communities were sampled from wounds in all groups 24 hours post-infection (n=6, all groups) and showed >95% Acinetobacter spp. community composition in all mice (FIG. 3, top row). By Day 3 post-infection, placebo treated mice (n=7) showed 5/7 mice having a wound composed of >95% Acinetobacter spp., whereas two mice had wounds with Enterobacter spp. present (>10% community composition) (FIG. 3, middle row). Rifampin-treated mice sampled at Day 3 (n=7) all had wounds composed of >95% Acinetobacter spp, while 6/7 wounds from doxycycline-treated mice (n=7) were composed of >95% Acinetobacter spp., with one wound having a significant (>10%) Enterobacter spp. presence. On Day 7, the placebo treated mice (n=6) all had wounds composed of >95% Acinetobacter spp, while the rifampin (n=6), and doxycycline (n=6) groups both had 5/6 wounds from mice comprised of >95% Acinetobacter spp while one wound from each treatment group were >60% Staphylococcus (FIG. 3, bottom row).

These results show that there was not significant contamination from other bacterial species utilizing the current methods employed in this study. Therefore, the necrotizing tissue damage and inflammation observed can be attributed to the A. baumannii that was inoculated into the wound bed. As the bacteria proliferated over time in the untreated mice, the increase in wound damage and the time to close can also be attributed to the increase in A. baumannii numbers.

### CFU Burden in Wound Tissue

An untreated group of mice (n=6) was used to assess the log CFU burden at the time of treatment (4 hours post-infection) showed 5.2×10⁵ CFU per 4 mm tissue biopsy punch (95% CI 4.4-5.8) (data not shown). After 24 hours of infection, placebo-treated mice (n=6) had a median log CFU burden of 6.7×10⁶, while rifampin-treated mice (n=6) had 1-log fewer with a median log CFU burden of 5.7×10⁵ (p<0.01) and doxycycline-treated mice had a median log CFU of 5.9×10⁵ (FIG. 4). Samples taken at 3 days post-infection revealed that placebo-treated mice (n=7) had a median log CFU burden of 7.0×10⁶ per punch, while both rifampin (n=8) and doxycycline (n=8)-treated groups decreased to 4.7×10⁴ (p<0.001) and 5.0×10⁴ (p<0.05), respectively. On Day 7, post-infection placebo treated mice (n=9) maintained a high median log CFU burden of 7.3×10⁶. While still significantly lower than the placebo-treated group, mice in both rifampin (n=9) and doxycycline (n=9) treated groups saw increases in median log CFU burden compared to their Day 3 values to 6.1×10⁵ (p<0.01) and 6.2×10⁵ (p<0.05), respectively.

### SEM Analysis of Biofilm Formation on Occlusive Dressings and Wound Tissue

Within 24 hours after inoculation, masses of bacteria where visible in punches taken from the wounds beds of mice in all treatment groups, while initial attachment was visible on occlusive dressing only in the placebo-treated groups and doxycycline-treated groups. There was no substantial amount of bacteria observed on the dressing removed one day post-infection in the rifampin-treated mice, and at one day post-infection, and no biofilm formation was observed on dressings on later days, Days 3 or 7 post-infection in the rifampin-treated group. By Day 3, the host-associated matrix proteins, red blood cells, and immune effectors can be observed in the wound bed of placebo-treated mice obfuscating the bacterial cells, while complex biofilm architecture and a continuous substratum of bacteria formed on the occlusive dressing. In both rifampin and doxycycline-treated mice, at Day 3 post-infection, the wound beds are rife with host-associated matrix proteins, blood cells, and immune effectors, similar to that of placebo-treated group. Doxycycline-treated mice appeared to have pockets of bacteria trapped underneath host-associated matrix on the occlusive dressings on Day 3, but lacked the continuous substratum or higher architecture of the biofilm seen in the placebo-treated group.

A wound infection model was developed utilizing A. baumannii as a sole infectious agent and that included multiple measurable outcomes with effect sizes in quantitative endpoints, which permitted small sample sizes for antimicrobial evaluation. Previous work had shown cyclophosphamide-induced neutropenia allowed A. baumannii to establish infections in a pulmonary model using smaller inoculums than in immunocompetant mice (12). A similar model was used to identify AB5075 as an isolate that could use as a reference strain for all subsequent studies (22), and in a similar immunocompromised background, infections with AB5075 resulted in wounds that took in excess of 21 days to close (FIG. 1). In contrast, uninfected wounds closed no later than 10 days post-infection and antibiotic-treated wounds closed between Day 15 to Day 21 (FIG. 1).

The retarded wound closure rate observed in the inventive model was accompanied by weight loss of up to 25% infection-day body weight two days post-infection (FIG. 2). Therefore, weight loss may be used as an indicator of A. baumannii infection in the inventive model. Gross pathology of infected wounds indicated tissue may begin to devitalize as soon as three days post-infection around the wound perimeter and remain visibly swollen until 15 days after inoculation with AB5075. An evaluation of the wound using standard histopathology showed inflammation throughout the large wound perimeter extending down to the spinal column by Day 7. Histopathology from infected mice on day 23 revealed wounds had re-epithelialized but showed little evidence of wound perimeter contraction. In order to investigate the dissemination of AB5075 throughout the wound bed, sections of tissue were fixed 24-hours post-infection and interrogated with Acinetobacter-specific PNA probes. The PNA probes revealed that the bacteria had spread rapidly beyond the wound bed through the underlying muscle and down to the spinal column. The A. baumannii appeared to localize in the interstitial space between muscle fibers and the interstitial space between epithelial cells.

The localization of A. baumannii in the interstitial space between cells is an interesting observation given previous reports of A. baumannii invading human epithelial cells in tissue culture experiments at low levels (38, 39, 40, 41). While there is agreement between groups that this invasion of tissue culture cells takes place (38), it is not clear if invasion occurs in vivo or in human patients. No intracellular bacteria were observed in any of the histopathology evaluation of mouse tissue, even at 100× maginifications (data not shown), so perhaps what laboratories are reporting as invasion is actually a “trapping event” in the interstitial space in between tissue culture cells; or the A. baumannii do invade host cells, even in vivo, but our histopathology observations cannot detect such a fleeting event. Invasion and attachment could also be strain dependent as was suggested in a number of reports (40, 41, 42), and perhaps because AB5075 disseminates better than other strains (22), it may also lack traits that potentially restrict dissemination such as a ChoP binding site identified in playing an important role in invasion (43), meaning the more a strain invades, perhaps the less it disseminates.

Regardless of the role invasion plays in vivo, the sequelae of infection observed post-inoculation are consistent to what is observed in skin and soft tissue infections of human patients. For example, the A. baumannii penetrated the layers of tissue from the initial wound bed inoculum all the way to bone tissue, and this is consistent with the tissue penetration observed in A. baumannii-infected patients (44) as well as a potential to cause osteomyelitis (45). Biopsy punches of infected tissue further revealed the CFU burden to have reached ˜1×10⁷ CFU/4 mm punch within 24 hours of infection (FIG. 4); this burden is maintained or exceeded on Day 3 and Day 7 post-infection. To ensure the infection could be attributed to AB5075 and not to other commensals or contaminant pathogens, a 16S community profiling was conducted in parallel to CFU enumeration. Nearly all samples taken from infected, but untreated wounds, showed a >95% dominance of Acinetobacter with only a minor fraction of communities being composed of Enterobacter in two mice from this group. It is hoped that the community profiling that showed overwhelming Acinetobacter presence in the wound is considered to be experimental evidence that implicates A. baumannii to be sole pathogen that does not require the presence of other bacterial species to drive infection. While immunosuppression is required to establish an infection, and it is agreed that A. baumannii is a mild pathogen when compared to other bacterial species; it is evident from the microbiome evaluation in this study that A. baumannii can establish a wound infection without other bacterial species being major contributors. However, it should also be noted, that the data does not indicate that the presence of other pathogens could still result in a more deleterious infection. In pilot studies, we also found that higher inoculums (CFU≧10⁶) resulted in sepsis, and unfortunately, animal death (data not shown), which also suggests that increased A. baumannii numbers can lead to more severe sequelae that did not require other bacterial species.

A final observation from this wound model of infection was the formation of robust biofilm within the wound bed and on the occlusive dressing above the wound bed. While it is not surprising that A. baumannii formed biofilms in this model given previous work showing the importance of biofilm (38, 40), it was somewhat surprising that AB5075 could achieve the levels of biofilm observed given that the strain does not make such robust biofilms in vitro (22). Therefore, perhaps there are cues in the in vivo environment that trigger biofilm assembly, and in particular this strain, that have not been discovered. Regardless, biofilm formation in the model provides another metric for antimicrobial efficacy. In fact, given that many laboratories are now developing anti-biofilm strategies (46), the SEM data provided in the implementation of this model can at least provide non-quantitative assessment of an anti-biofilm product.

The murine wound infection model of this invention uses all of the endpoints mentioned above to provide a robust data set for evaluating an infection and antimicrobials used for treatment. It is a powerful tool to evaluate new antimicrobials for not just A. baumannii, but all difficult bacterial infections, such as those caused by the MDR-ESKAPE pathogens, as well as those cause by other wound infection pathogens. While in this study, only a systemic application was shown, the model has also been used to determine the efficacy of new topical antimicrobial treatments against A. baumannii, K. pneumoniae, MRSA, and P. aeruginosa (unpublished results). Bioluminescent strains can be utilized to monitor bacterial numbers without sacrificing animals. Therefore, the utilization of such a model could be an important first, preclinical step when determining the efficacy of antibiotics in development for skin and soft tissue infections caused by any bacterial pathogen.

### Example 3

### Evaluation of Therapeutics Against a Multi-Drug Resistant Strain of Klebsiella Pneumoniae in a Murine Wound Model of Infection

Klebsiella pneumoniae is often isolated and reported to be the cause of these infections, and if untreated, dissemination into the bloodstream can lead to further morbidity and possible mortality. K. pneumoniae isolates are also becoming more multidrug-resistant (MDR) making many antibiotic treatments ineffective. In this study, an alternative to antibiotics for the treatment for wound infections was tested in the murine model of wound infection. A topical gallium formulations, was applied to the inventive murine model and tested for efficacy against K. pneumoniae.

### Bacterial Strain and Inocula Preparation.

K pneumoniae clinical isolate KP4640 was used in all experiments. KP4640 was propagated on Lennox lysogeny broth (LB) (Becton, Dickinson and Co., Sparks, Md.) for all experiments. To prepare inocula for animal infection, 100 μl of a KP4640 overnight culture was subcultured into 10 mL of LB, and then grown at 37° C. and shaking at 250 rpm in a 250 ml Erlenmeyer flask. Cells in mid-exponential growth phase were harvested at an OD600 of 0.8. Cells were washed twice with sterile phosphate buffered saline (PBS) and then resuspended in PBS so that 25 μL of the suspension contained 1×106 cells. The cell concentration of the suspension was verified via a Petroff-Hauser counting chamber prior to inoculation of mice, and confirmed by serial dilution and plating on LB agar using a spiral plating system (AUTOPLATE®, Advanced Instruments, Inc., Norwood, Mass.).

### Murine Dorsal Wound Model

Female BALB/c mice were purchased from the National Cancer Institute, Animal Production Program (Frederick, Md.). The mice used in these experiments were six to ten weeks of age and weighed 14 to 20 g. Each mouse received 150 mg/kg and 100 mg/kg cyclophosphamide intraperitoneal (IP) injections, before wounding and infection, on days −4 and −1, respectively. On Day 0, the day of wounding and inoculation, mice were anesthetized with ketamine 130 mg/kg (KETASET®, Fort Dodge Animal Health, Fort Dodge, Iowa), xylazine 10 mg/kg (ANASED®, Lloyd Inc. Shenandoah, Iowa), and buprenorphine 0.05 mg/kg (Hospira Inc., Lake Forest, Ill.) injections were given for pain management. Hair was clipped from the cervical to mid-lumbar dorsum, and the skin scrubbed with iodine solution followed by an ethanol rinse. A 6.0 mm disposable skin biopsy punch (VisiPunch, Huot Instruments, LLC, Menomonee Falls, Wis.) was used to create a full-thickness skin defect overlying thoracic spinal column and the adjacent musculature. Twenty-five μL containing 1×10⁶ KP4640 cells in a PBS suspension were pipetted into the wound and allowed to absorb for three minutes. A circular cutout (30 mm diameter) of transparent dressing (TEGADERM™ Roll, 3M Health Care, St. Paul, Minn.) was placed over the wound and secured with tissue adhesive (VETBOND™, 3M Animal Care, St. Paul, Minn.).

For experiments assessing efficacy of the gallium treatment, beginning four hours post-inoculation, mice were treated with either 25 uL of HEC placebo either once or twice daily, and 25 uL of HEC with 0.1% w/v gallium formulation twice-daily, or HEC with 0.3% w/v gallium formulation once-daily. On Day 3 the transparent dressing was removed, treatment discontinued, and the wound monitored for closure through Day 15 for wound closure studies, while dressings were removed on day 7 for mice monitored for biofilm development. In experiments investigating wound CFU burden, treatments began 4 hours post-infection and mice were treated once daily with either placebo HEC, 0.1% gallium formulation w/v in HEC, or 0.3% gallium formulation w/v in HEC.

Gross pathology and histopathology revealed that K. pneumoniae infected wounds appeared to close faster with 0.1% or 0.3% treatments of topical gallium formulation accompanied by less inflammation when compared to untreated controls. Similarly, quantitative indications of infection remediation such as weight loss and wound area suggested that treatment with topical gallium formulation improved healing and reduced bacterial burden when compared to untreated controls. Bacterial burden was measured one day and three days following inoculation, and a 0.5-1.5 log reduction of colony forming units was observed. Lastly, upon scanning electron microscopy analysis, biofilms on dressings dispersed with gallium treatment when compared to untreated controls. These results suggest that a topical application of topical gallium formulation will reduce biofilm and lessen the bacterial load of K. pneumoniae, which in turn, can promote wound healing.

### REFERENCES

1. Michalopoulos A, Falagas M E. 2010. Treatment of Acinetobacter infections. Expert Opin. Pharmacother. 11:779-788.
2. Dallo S F, Weitao T. 2010. Insights into Acinetobacter war-wound infections, biofilms, and control. Adv. Skin Wound Care. 23:169-174.
3. Grosso F, Quinteira S, Peixe L. 2010. Emergence of an extreme-drug-resistant (XDR) Acinetobacter baumannii carrying blaOXA-23 in a patient with acute necrohaemorrhagic pancreatitis. J. Hosp. Infect. 75:82-83.
4. Gordon N C, Wareham D W. 2009. A review of clinical and microbiological outcomes following treatment of infections involving multidrug-resistant Acinetobacter baumannii with tigecycline. J. Antimicrob. Chemother. 63:775-780.
5. Munoz-Price L S, Zembower T, Penugonda S, Schreckenberger P, Lavin M A, Welbel S, Vais D, Baig M, Mohapatra S, Quinn J P, Weinstein R A. 2010. Clinical outcomes of carbapenem-resistant Acinetobacter baumannii bloodstream infections: study of a 2-state monoclonal outbreak. Infect. Control Hosp. Epidemiol. 31:1057-1062.
6. Keen E F 3rd, Robinson B J, Hospenthal D R, Aldous W K, Wolf S E, Chung K K, Murray C K. 2010. Prevalence of multidrug-resistant organisms recovered at a military burn center. Burns. 36:819-825.
7. Sheppard F R, Keiser P, Craft D W, Gage F, Robson M, Brown T S, Petersen K, Sincock S, Kasper M, Hawksworth J, Tadaki D, Davis T A, Stojadinovic A, Elster E. 2010. The majority of US combat casualty soft-tissue wounds are not infected or colonized upon arrival or during treatment at a continental US military medical facility. Am. J. Surg. 200:489-495.
8. Boyanova L, Mitov I. 2013. Antibiotic resistance rates in causative agents of infections in diabetic patients: rising concerns. Expert Rev. Anti. Infect. Ther. 11:411-420.
9. Furniss D, Gore S, Azadian B, Myers S R. 2005. Acinetobacter infection is associated with acquired glucose intolerance in burn patients. J. Burn Care Rehabil. 26:405-408.
10. Corradino B, Toia F, di Lorenzo S, Cordova A, Moschella F. 2010. A difficult case of necrotizing fasciitis caused by Acinetobacter baumannii. Int J Low Extrem Wounds. 9:152-154.
11. Montero A, Ariza J, Corbella X, Doménech A, Cabellos C, Ayats J, Tubau F, Ardanuy C, Gudiol F. 2002. Efficacy of colistin versus beta-lactams, aminoglycosides, and rifampin as monotherapy in a mouse model of pneumonia caused by multiresistant Acinetobacter baumannii. Antimicrob. Agents Chemother. 46:1946-1952.
12. Eveillard M, Soltner C, Kempf M, Saint-André J P, Lemarié C, Randrianarivelo C, Seifert H, Wolff M, Joly-Guillou M L. 2010. The virulence variability of different Acinetobacter baumannii strains in experimental pneumonia. J. Infect. 60:154-161.
13. Breslow J M, Monroy M A, Daly J M, Meissler J J, Gaughan J, Adler M W, Eisenstein T K. 2011. Morphine, but not trauma, sensitizes to systemic Acinetobacter baumannii infection. J. Neuroimmune Pharmacol. 6:551-565.
14. Luo G, Spellberg B, Gebremariam T, Bolaris M, Lee H, Fu Y, French S W, Ibrahim A S. 2012. Diabetic murine models for Acinetobacter baumannii infections. J. Antimicrob. Chemother. 67:1439-1445.
15. Russo T A, Beanan J M, Olson R, MacDonald U, Luke N R, Gill S R, Campagnari A A. 2008. Rat pneumonia and soft-tissue infection models for the study of Acinetobacter baumannii biology. Infect Immun. 76:3577-3586.
16. Gaddy J A, Arivett B A, McConnell M J, López-Rojas R, Pachón J, Actis L A. 2012. Role of acinetobactin-mediated iron acquisition functions in the interaction of Acinetobacter baumannii strain ATCC 19606T with human lung epithelial cells, Galleria mellonella caterpillars, and mice. Infect Immun. 80:1015-1024.
17. Cirioni O, Silvestri C, Ghiselli R, Orlando F, Riva A, Gabrielli E, Mocchegiani F, Cianforlini N, Trombettoni M M, Saba V, Scalise G, Giacometti A. 2009. Therapeutic efficacy of buforin II and rifampin in a rat model of Acinetobacter baumannii sepsis. Crit. Care Med. 37:1403-1407.
18. Shankar R, He L K, Szilagyi A, Muthu K, Gamelli R L, Filutowicz M, Wendt J L, Suzuki H, Dominguez M. 2007. A novel antibacterial gene transfer treatment for multidrug-resistant Acinetobacter baumannii-induced burn sepsis. J. Burn Care Res. 28:6-12.
19. Mihu M R, Sandkovsky U, Han G, Friedman J M, Nosanchuk J D, Martinez L R. 2010. The use of nitric oxide releasing nanoparticles as a treatment against Acinetobacter baumannii in wound infections. Virulence. 1:62-67.
20. Dai T, Tegos G P, Lu Z, Huang L, Zhiyentayev T, Franklin M J, Baer D G, Hamblin M R. 2009. Photodynamic therapy for Acinetobacter baumannii burn infections in mice. Antimicrob. Agents Chemother. 53:3929-3934.
21. DeLeon K, Balldin F, Watters C, Hamood A, Griswold J, Sreedharan S, Rumbaugh K P. 2009. Gallium maltolate treatment eradicates Pseudomonas aeruginosa infection in thermally injured mice. Antimicrob. Agents Chemother. 53:1331-1337.
22. Black C C, Thompson M G, Penwell W F, Kessler J L, Clark L P, Jacobs A C, McQueary C N, Si Y, Gancz H Y, Honnold C L, Wise M C, Moon J K, Owen M T, Hallock J D, Kwak Y I, Summers A, Li C Z, Waterman P E, Lesho E P, Stewart R L, Rasko D A, Actis L A, Craft D W, Palys T J, Zurawski D V. 2013. Enhanced dissemination and sepsis elucidate the superior virulence of AB5075, a multidrug-resistant Acinetobacter baumannii isolate. mBio. In press.
23. Zurawski D V, Thompson M G, McQueary C N, Matalka M N, Sahl J W, Craft D W, Rasko D A. 2012. Genome sequences of four divergent multidrug-resistant Acinetobacter baumannii strains isolated from patients with sepsis or osteomyelitis. J. Bacteriol. 194:1619-1620.
24. Zuluaga, A F, Salazar B E, Rodriguez C A, Zapata A X, Agudelo M, Vesga O. 2006. Neutropenia induced in outbred mice by a simplified low-dose cyclophosphamide regimen: characterization and applicability to diverse experimental models of infectious diseases. BMC Infect. Dis. 6:55.
25. Pachón-Ibáñez ME, Docobo-Pérez F, López-Rojas R, Domínguez-Herrera J, Jiménez-Mejias M E, García-Curiel A, Pichardo C, Jiménez L, Pachón J. 2010. Efficacy of rifampin and its combinations with imipenem, sulbactam, and colistin in experimental models of infection caused by imipenem-resistant Acinetobacter baumannii. Antimicrob. Agents Chemother, 54:1165-1172.
26. Rodriguez-Hernandez M J, Pachon J, Pichardo C, Cuberos L, Ibanez-Martinez J, Garcia-Curiel A, Caballero F J, Moreno I, Jiménez-Mejías M E. 2000. Imipenem, doxycycline and amikacin in monotherapy and in combination in Acinetobacter baumannii experimental pneumonia. J. Antimicrob. Chemother. 45:493-501.
27. Schloss P D, Westcott S L, Ryabin T, Hall J R, Hartmann M, Hollister E B, Lesniewski R A, Oakley B B, Parks D H, Robinson C J, Sahl J W, Stres B, Thallinger G G, Van Horn D J, Weber C F. 2009. Introducing mothur: open-source, platform-independent, community-supported software for describing and comparing microbial communities. Appl. Environ. Microbiol. 75:7537-7541.
28. Pruesse E, Quast C, Knittel K, Fuchs B M, Ludwig W, Peplies J, Glöckner F O. 2007. SILVA: a comprehensive online resource for quality checked and aligned ribosomal RNA sequence data compatible with ARB. Nucleic Acids Res. 35:7188-7196.
29. Edgar R C, Haas B J, Clemente J C, Quince C, Knight R. 2011. UCHIME improves sensitivity and speed of chimera detection. Bioinformatics. 27:2194-2200.
30. Huse S M, Welch D M, Morrison H G, Sogin M L. 2010. Ironing out the wrinkles in the rare biosphere through improved OTU clustering. Environ. Microbiol. 12:1889-1898.
31. Wang Q, Garrity G M, Tiedje J M, Cole J R. 2007. Naive Bayesian classifier for rapid assignment of rRNA sequences into the new bacterial taxonomy. Appl. Environ. Microbiol. 73:5261-5267.
32. Werner J J, Koren O, Hugenholtz P, DeSantis T Z, Walters W A, Caporaso J G, Angenent L T, Knight R, Ley R E. 2012. Impact of training sets on classification of high-throughput bacterial 16s rRNA gene surveys. ISME J. 6:94-103.
33. Malic, S., et al. 2009. Detection and identification of specific bacteria in wound biofilms using peptide nucleic acid fluorescent in situ hybridization (PNA FISH). Microbiology. 155:2603-2611.
34. Prabhakara R, Harro J M, Leid J G, Keegan A D, Prior M L, Shirtliff M E. 2011. Suppression of the inflammatory immune response prevents the development of chronic biofilm infection due to methicillin-resistant Staphylococcus aureus. Infect. Immun. 79:5010-5018.
35. Gabaglia C R, Pedersen B, Hitt M, Burdin N, Sercarz E E, Graham F L, Gauldie J, Braciak T A. 1999. A single intramuscular injection with an adenovirus-expressing IL-12 protects BALB/c mice against Leishmania major infection, while treatment with an IL-4-expressing vector increases disease susceptibility in B10.D2 mice. J. Immunol. 162:753-760.
36. Boucher H W, Talbot G H, Bradley J S, Edwards J E, Gilbert D, Rice L B, Scheld M, Spellberg B, Bartlett J. 2009. Bad bugs, no drugs: no ESKAPE! An update from the Infectious Diseases Society of America. Clin. Infect. Dis. 48:1-12.
37. Hoffman M, Harger A, Lenkowski A, Hedner U, Roberts H R, Monroe D M. 2006. Cutaneous wound healing is impaired in hemophilia B. Blood. 108:3053-3060.
38. Brossard K A, Campagnari A A. 2012. The Acinetobacter baumannii biofilm-associated protein plays a role in adherence to human epithelial cells. Infect. Immun. 80:228-233.
39. Jacobs A C, Hood I, Boyd K L, Olson P D, Morrison J M, Carson S, Sayood K, Iwen P C, Skaar E P, Dunman P M. 2010. Inactivation of phospholipase D diminishes Acinetobacter baumannii pathogenesis. Infect. Immun. 78:1952-1962.
40. Gaddy J A, Tomaras A P, Actis L A. 2009. The Acinetobacter baumannii 19606 OmpA protein plays a role in biofilm formation on abiotic surfaces and in the interaction of this pathogen with eukaryotic cells. Infect. Immun. 77:3150-3160.
41. Choi C H, Lee J S, Lee Y C, Park T I, Lee J C. 2008. Acinetobacter baumannii invades epithelial cells and outer membrane protein A mediates interactions with epithelial cells. BMC Microbiol. 8:216.
42. Giannouli M, Antunes L C, Marchetti V, Triassi M, Visca P, Zarrilli R. 2013. Virulence-related traits of epidemic Acinetobacter baumannii strains belonging to the international clonal lineages I-III and to the emerging genotypes ST25 and ST78. BMC Infect. Dis. 13:282.
43. Smani Y, Docobo-Pérez F, López-Rojas R, Domínguez-Herrera J, Ibáñez-Martínez J, Pachón J. Platelet-activating factor receptor initiates contact of Acinetobacter baumannii expressing phosphorylcholine with host cells. J. Biol. Chem. 287:26901-26910.
44. Eberle B M, Schnüriger B, Putty B, Barmparas G, Kobayashi L, Inaba K, Belzberg H, Demetriades D. 2010. The impact of Acinetobacter baumannii infections on outcome in trauma patients: a matched cohort study. Crit. Care Med. 38:2133-2138.
45. Yun H C, Branstetter J G, Murray C K. 2008. Osteomyelitis in military personnel wounded in Iraq and Afghanistan. J. Trauma. 64:S163-168.
46. Worthington R J, Richards J J, Melander C. 2012. Small molecule control of bacterial biofilms. Org. Biomol. Chem. 10:7457-7474.
47. Pendleton J N, Gorman S P, Gilmore B F. 2013. Clinical Relevance of the ESKAPE Pathogens. Expert Rev Anti Infect Ther. 2013.11(3):297-308.
48. Martin, Remington et al., Pharmaceutical Sciences, 15th Ed., Mack Publ. Co., Easton, Pa. (1975).
49. Mok C C, Wong R W, Lai K N. Treatment of severe proliferative lupus nephritis: the current state. Ann Rheum Dis 62, 799-804 (2003).
50. Laccarino et al., Mycophenolate mofetil: what is its place in the treatment of autoimmune rheumatic diseases? Autoimmunity Reviews 6, 190-195 (2007).

What is claimed is:

1) A method producing a murine wound model for infection, comprising:
a) administering to an small animal a therapeutically effective amount of an immunosuppressive drug day −5 to day −1 prior to pathogen inoculation (day 0);
b) creating one or more cutaneous wound on the animal;
c) inoculating the wound sites with an virulent strain of a pathogen associated with wound infection; and
d) allowing said wound infection to occur.

2) The method of claim 1, wherein said immunosuppressive drug is selected from the group consisting of cyclophosphamide (CYC), azathioprine (AZA), cyclosporine A (CSA), and mycophenolate mofetil (MMF), prednisone, methyl prednisolone, monoclonal antibodies against T cells, antilymphocyte globulin and antithymocyte globulin.

3) The method of claim 1, wherein said immunosuppressant drug is administered to the small animal in one or more doses.

4) The method of claim 1, wherein said wound infection pathogen is selected from the group consisting of ESKAPE pathogen, Escherichia coli, Stenotrophomonas maltophilia, Streptococcus, Corynebacterium, Candida albicans, Aspergillus and Mucosales species.

5) The method of claim 4, wherein said ESKAPE pathogen is selected from the group consisting of Enterococcus faecium, Staphylococcus aureus, Klebsiella pneumoniae, Acinetobacter baumannii, Pseudomonas aeruginosa, Enterobacter spp.

6) The method of claim 1, further comprising e) covering the wound with a dressing.

7) The method of claim 5, wherein said wound infection pathogen is A. baumannii clinical isolate AB5075 or K. pneumoniae clinical isolate KP4640.

8) The method of claim 2, wherein said immunosuppressive drug is cyclophosphamide (CYC).

9) The method of claim 8, wherein said cyclophosphamide is administered on Day −4 at 150 mg/kg and Day −1 at 100 mg/kg (day 0 is the day of pathogen inoculation).

10) The method of claim 8, wherein said cyclophosphamide may be administered via intraperitoneal injection.

11) The method of claim 1, wherein said small animal is a rat, mouse, hamster, or gerbil.

12) A method for testing virulence of a wound pathogen, comprising
a) producing a murine wound model according to method of claim 1, with inoculation of an interested pathogen in step b) of claim 1; and
b) determining the virulence of said pathogen.

13) The method of claim 12, wherein said virulence is determined by the length of the infection, tissue disruption or damage and bacterial burden.

14) A method for evaluating efficacy of an wound healing therapeutic, comprising
a) producing a murine wound model using method of claim 1, by inoculating said animal of with one or more pathogen associated with wound infection in step b) of claim 1;
b) administering an pharmaceutically effective amount of said therapeutics to said animal; and
c) evaluating efficacy of said wound healing therapeutic against said target pathogen.

15) The method of claim 14, wherein said wound healing therapeutic consist of a treatment, a drug, a vaccine antimicrobial agent, and a combination thereof,

16) The method of claim 15, wherein said wound healing therapeutic prevents an infection by one or more wound infection pathogen.

17) The method of claim 16, wherein said vaccine is administered to the animal prior to step b) of claim 1.

18) The method of claim 15, wherein said is administered to the animal after step b) of claim 1.

19) The method of claim 15, wherein said wound healing therapeutic is administered with a pharmaceutical carrier or an adjuvant.

20) The method of claim 13, wherein the efficacy of said wound healing therapeutic is evaluated using one or more tests selected from the group consisting of
a) genetic analysis;
b) PNA-fish analysis;
c) biofilm assays and visualization;
d) bacteria load study; and
e) histological and immunological analysis.
